(19) United States
(12) Patent Application Publication
(10) Pub. No.: US 2026/0201145 A1
(43) Pub. Date: Jul. 16, 2026

(54) PROCESS FOR THE PREPARATION OF AN ELASTOMER COMPOSITION COMPRISING A LINEAR OR CYCLIC DI-CARBOXYLIC ACID

(71) Applicant: Pirelli Tyre S.p.A., Milano (IT)

(72) Inventors: Maurizio Stefano GALIMBERTI, Milano (IT); Vincenzina BARBERA, Milano (IT); Fatima MARGANI, Milano (IT); Luca GIANNINI, Milano (IT); Silvia GUERRA, Milano (IT)

(73) Assignee: Pirelli Tyre S.p.A., Milano (IT)

(21) Appl. No.: 19/136,566

(22) PCT Filed: Dec. 13, 2023
(86) PCT No.: PCT/IB2023/062594

§ 371 Date: Jun. 6, 2025

(30) Foreign Application Priority Data
Dec. 14, 2022 (IT) 102022000025608

(51) Int. Cl. C08K 5/1545; C07D 309/36; C08L 7/00

(52) U.S. Cl. CPC C08K 5/1545; C07D 309/36; C08L 7/00; C08L 2312/00

(57) ABSTRACT

The present invention relates to an improved process for the preparation of an elastomer composition comprising a linear or cyclic di-carboxylic acid, or a derivative thereof.

### FIELD OF THE INVENTION

The present invention relates to an improved process for the preparation of an elastomer composition comprising a linear or cyclic di-carboxylic acid, or a derivative thereof.

### PRIOR ART

The peculiar feature of elastomeric composites is the entropic elasticity, which occurs above the glass transition temperature of the elastomers and is at the basis of their performances. Diene elastomers, i.e. elastomers with unsaturation in the backbone chain such as poly(1,4-cis-butadiene) and poly(1,4-cis-isoprene), are characterized by a great mobility of the backbone chain, due to the easy rotation of the polymer chain around the single bonds close to the unsaturations. Upon crosslinking the polymer chains of these unsaturated elastomers, the composite acquires the property of entropic elasticity. However, despite the outstanding elasticity, elastomeric composites do not have the mechanical properties required for demanding applications, such as the one in tyre compounds. To achieve such properties, the composites have to be loaded with reinforcing fillers.

Since the beginning of the twentieth century, carbon black has been used as reinforcing filler for elastomeric composites. Thanks to the addition of carbon black, both the static and dynamic-mechanical properties of elastomeric composites increase. However, carbon black brings also about a remarkable increase of the hysteresis, hence of the dissipation of energy, of the elastomeric composites. Indeed it is known that the elastic modulus of a composite material filled with carbon black, to which sinusoidal stresses have been applied, decreases, passing from minimum strain up to around 25% of strain (limit estimated for linear behaviour). This phenomenon is known as the “Payne Effect” and is an indicator of the energy dissipation of the material.

To have mechanical reinforcement and low dissipation of energy, silica is used as reinforcing filler, in place of or together with carbon black. Thanks to the use of a coupling agent, typically a silane containing sulphur atoms, a chemical bond is established between silica and the elastomers' chains, and this leads to the reduction of hysteresis and of the dissipation of energy. Hence, the use of silica has been dramatically increasing over the last decades, despite the drawbacks connected to its use.

Indeed, silica leads to the increase of the compound viscosity, to the worsening of processability and to the shortening of the storage time of the composites. Such drawbacks arise from the surface activity of silica: the polar groups promote extensive supramolecular interactions. Of note, a shorter storage time results in the need for a specific planning for the compounds' production and the procedures for storing and moving the compounds, with a clear impact on the logistics.

Moreover, to achieve efficient mixing of silica-based composites, specific expensive mixing equipment are required. Due to the use of a silane, silica-based compounds have increased adhesiveness to the metal parts of the mixing machines, and this requires special treatments of the metal surfaces. Finally, silica is corrosive and abrasive and this as well requires special treatments of the metal surfaces and the revision of the maintenance procedures.

These drawbacks are relevant at the industrial scale, for example when silica based elastomeric composites are used for tyre compounds. However, tyre compounds, particularly those used for tyre treads, are typically almost exclusively based on silica as the reinforcing filler and increasing research efforts are made to use silica in partial replacement of carbon black, also in tyre compounds other than the tyre tread. Research efforts are also made to use silica as the only filler in tyre compounds other than the tyre tread. This is because the prevailing objective is the reduction of energy dissipation of a rolling tyre and this objective has never been achieved, at least in the existing prior art, with carbon black as the sole reinforcing filler.

Indeed, several attempts at reducing the Payne effect due to carbon black have been made, for example by optimizing its dispersion in the elastomer matrix, separating the aggregates and/or the elemental particles from it, covering them with a layer of elastomer. The Applicants have already reported different approaches to achieve this by the use of pre-formed adducts between sp² hybridized carbon allotropes and pyrrole derivatives or other suitable compounds (see e.g. WO 2016/050887, WO 2020/225595A1, WO 2020/222103). However no attempts at partially, or completely, replacing silica have been reported so far.

Another crucial problem is that the reaction between an elastomer composition, e.g. a tyre compound, in normal operating conditions, with oxygen and ozone leads to the modification of the chemical structure of the polymer chains. The reaction with oxygen leads to the introduction in the polymer chain of oxygenated functional groups, which badly affect the properties of the elastomers. The reaction with ozone is particularly detrimental as it leads to chain scission.

Quinolines, heterocyclic aromatic organic compounds containing a nitrogen atom, are commonly used as antioxidants in tyre compounds. A typical example of a quinoline used for these applications is 1,2-dihydro-2,2,4-trimethylquinoline (TMQ), an oil-based substance classified as acutely toxic if swallowed and causing serious eye irritation.

Paraphenylene diamines, organic compounds containing either primary or secondary or tertiary amino groups are typically used as antioxidant and anti-ozone agent. The most used paraphenylene diamine in tyre compounds is N-(1,3-dimethylbutyl)-N′-phenyl-p-phenylenediamine (6PPD), a known sensitizing agent which is harmful if swallowed and may cause an allergic skin reaction.

### SUMMARY OF THE INVENTION

The Applicants addressed the need for a process for the preparation of elastomer compositions overcoming, at least in part, the drawbacks described above.

In particular, the Applicants have tackled the problem of providing elastomer compositions able to achieve a balance between the advantages brought about by both silica and carbon black, while at the same time overcoming the related drawbacks. Moreover, the Applicants have tackled the problem of providing elastomer compositions effectively protected from the reaction with oxygen and ozone with a reduced content of toxic and harmful chemicals.

The Applicants have also addressed the problem to obtain such elastomer compositions by means of simple techniques, with reduced environmental impact, and using the standard solid state mixing techniques used in the tyres industry.

Surprisingly, the Applicants have found that these results can be achieved by a simplified process, relying on the addition of a linear or cyclic di-carboxylic acid, or a derivative thereof, directly within the elastomer composition, thus avoiding the preparation of a pre-formed adduct with a sp² hybridized carbon allotrope as described for example in WO2020/222103 (two-step process) mentioned above. By this process, the Applicants have obtained elastomer compositions with optimal dynamic-mechanical properties, at least comparable to those typically obtained by the two-step preparation and to those obtained using silica as reinforcing filler, and at the same time the processability and the abovementioned advantages linked to the use of carbon black.

Furthermore the Applicants have surprisingly found that the linear or cyclic di-carboxylic acids, or derivatives thereof, suitable for the direct preparation of elastomer compositions may also provide antioxidant and anti-ozone properties, thus replacing more toxic chemical compounds typically used as antioxidant and anti-ozone agents.

The process according to the present invention allows to obtain elastomer compositions exhibiting low hysteresis and a reduced Payne effect, with consequent reduced dissipation of energy, at the same time endowed with prolonged resistance to the action of oxygen and ozone, and particularly suitable as compounds for tyres.

According to a first aspect, the invention relates to a process for the preparation of an elastomer composition comprising a compound represented by formula (I) below:

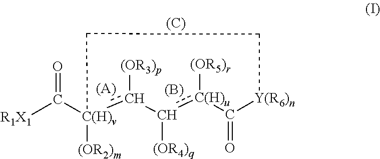

wherein the compound of formula (I) can be linear or cyclic and the symbol [symbol] does not represent a bond when the compound is linear, and it represents a bond when the compound is cyclic; and
m, v, p, q, r, u, and n can independently be either equal to 1 or equal to 0; and
X₁ and Y can be O or NR₇;
and wherein
R₇ is selected from the group consisting of: hydrogen, C₂-C₆ acyl, C₁-C₆ alkyl, linear or branched C₂-C₆ alkenyl or alkynyl, aryl, linear or branched C₁-C₆ alkyl aryl, linear or branched C₂-C₆ alkenyl aryl, linear or branched C₂-C₆ alkynyl-aryl, heteroaryl; and
if X₁ and Y are NR₇ then:
   R₁, R₆ are independently selected from the group consisting of: hydrogen, linear or branched C₁-C₆ alkyl, linear or branched C₂-C₆ alkenyl or alkynyl, aryl, linear or branched C₁-C₆ alkyl aryl, linear or branched C₂-C₆ alkenyl aryl, linear or branched C₂-C₆ alkynyl-aryl, heteroaryl;
if X₁ and Y are O then:
   R₁, R₆ are independently selected from the group consisting of: hydrogen, linear or branched C₁-C₆ alkyl, linear or branched C₂-C₆ alkenyl or alkynyl, aryl, linear or branched C₁-C₆ alkyl aryl, linear or branched C₂-C₆ alkenyl aryl, linear or branched C₂-C₆ alkynyl-aryl, heteroaryl, alkali metals, alkaline earth metals, transition metals, or

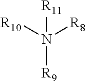

wherein R₈, R₉, R₁₀, R₁₁ are independently selected from the group consisting of: hydrogen, linear or branched C₁-C₆ alkyl, linear or branched C₁-C₆ alkyl aryl, linear or branched C₂-C₆ alkenyl aryl, linear or branched C₂-C₆ alkynyl-aryl, heteroaryl;
and wherein
R₂, R₃, R₄, R₅ are independently selected from the group consisting of: hydrogen, C₂-C₆ acyl, C₁-C₆ alkyl, linear or branched C₂-C₆ alkenyl or alkynyl, aryl, linear or branched C₁-C₆ alkyl aryl, linear or branched C₂-C₆ alkenyl aryl, linear or branched C₂-C₆ alkynyl-aryl, heteroaryl;
and wherein:
   when the compound of formula (I) is linear:
   n is 1 and v and/or m are 1;
   the symbols [symbol] and [symbol] independently represent a single or a double bond, and
   if the symbol [symbol] is a double bond, p and v are 0 and m is 1 or p and m are 0 and v is 1;
   if the symbol [symbol] is a double bond q and r are 0 and u is 1 or q and u are 0 and r is 1;
   if the symbol [symbol] is a single bond p, v and m are 1;
   if the symbol [symbol] is a single bond q, r and u are 1; and
when the compound of formula (I) is cyclic:
   the symbols [symbol] and [symbol] are a double bond, and m, v, p, q, n and u are 0 and r is 1, or m, v, p, q, n and r are 0 and u is 1;
said process comprising the steps of:
   a) providing one or more elastomers, a compound of formula (I), and at least one reinforcing filler; and
   b) performing at least one mixing step.

Of note, in step a) the compound of formula (I) is provided as is, together with at least one or more elastomers. Thus, in the process according to the present invention, no premixing step between the compound of formula (I) and said at least one reinforcing filler is performed.

### BRIEF DESCRIPTION OF THE FIGURES

The description is illustrated here with reference to the attached drawings, provided solely by way of example and not limiting the invention.

FIG. 1 shows the ¹H NMR spectrum (DMSO-d₆, 400 MHz) of 3,4,5-triacetoxy-6-oxo-tetrahydro-pyran-2-carboxylic acid (precursor) obtained as disclosed in example 1.

FIG. 2 shows the ¹H NMR spectrum (DMSO-d₆, 400 MHz) of 3-hydroxy-2-oxo-2H-pyran-6-carboxylic acid (compound 2) obtained as disclosed in example 2.

FIG. 3 shows the ¹H NMR spectrum (DMSO-d₆, 400 MHz) of ethyl 3-hydroxy-2-oxo-2H-pyran-6-carboxylate (compound 3) obtained as disclosed in example 3.

### DETAILED DESCRIPTION OF THE INVENTION

### Definitions

According to the present description, the terms “carbon allotrope” and “carbon-based filler” are used interchangeably, and/or are both indicated with the abbreviation CA.

For the purposes of the present description and the following claims, the compounds of formula (I) and (II) herein disclosed also include derivatives, such as esters, salts, enantiomers, diastereomers, preferably esters and salts.

In the following, the term “elastomer composition” is used to include the totality of all the components which are added in the preparation of the elastomer mixture, independently of the fact that these may all actually be present simultaneously, that they may be introduced sequentially or that they may then be traceable in the elastomer mixture or in the final tyre.

For the purposes of the present description and the following claims, the term “phr” (parts per hundreds of rubber) indicates the parts by weight of a defined component of the elastomer composition per 100 parts by weight of the elastomer polymer.

### DETAILED DESCRIPTION

According to a first aspect, the invention relates to a process for the preparation of an elastomer composition comprising a compound represented by formula (I) below:

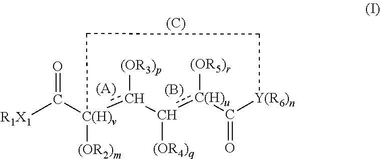

wherein the compound of formula (I) can be linear or cyclic and the symbol [symbol] does not represent a bond when the compound is linear, and it represents a bond when the compound is cyclic; and
m, v, p, q, r, u, and n can independently be either equal to 1 or equal to 0; and
X₁ and Y can be O or NR₇;
and wherein
R₇ is selected from the group consisting of: hydrogen, C₂-C₆ acyl, C₁-C₆ alkyl, linear or branched C₂-C₆ alkenyl or alkynyl, aryl, linear or branched C₁-C₆ alkyl aryl, linear or branched C₂-C₆ alkenyl aryl, linear or branched C₂-C₆ alkynyl-aryl, heteroaryl; and
if X₁ and Y are NR₇ then:
   R₁, R₆ are independently selected from the group consisting of: hydrogen, linear or branched C₁-C₆ alkyl, linear or branched C₂-C₆ alkenyl or alkynyl, aryl, linear or branched C₁-C₆ alkyl aryl, linear or branched C₂-C₆ alkenyl aryl, linear or branched C₂-C₆ alkynyl-aryl, heteroaryl;
if X₁ and Y are O then:
   R₁, R₆ are independently selected from the group consisting of: hydrogen, linear or branched C₁-C₆ alkyl, linear or branched C₂-C₆ alkenyl or alkynyl, aryl, linear or branched C₁-C₆ alkyl aryl, linear or branched C₂-C₆ alkenyl aryl, linear or branched C₂-C₆ alkynyl-aryl, heteroaryl, alkali metals, alkaline earth metals, transition metals, or

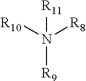

wherein R₈, R₉, R₁₀, R₁₁ are independently selected from the group consisting of: hydrogen, linear or branched C₁-C₆ alkyl, linear or branched C₁-C₆ alkyl aryl, linear or branched C₂-C₆ alkenyl aryl, linear or branched C₂-C₆ alkynyl-aryl, heteroaryl;
and wherein
R₂, R₃, R₄, R₅ are independently selected from the group consisting of: hydrogen, C₂-C₆ acyl, C₁-C₆ alkyl, linear or branched C₂-C₆ alkenyl or alkynyl, aryl, linear or branched C₁-C₆ alkyl aryl, linear or branched C₂-C₆ alkenyl aryl, linear or branched C₂-C₆ alkynyl-aryl, heteroaryl;
and wherein:
   when the compound of formula (I) is linear:
   n is 1 and v and/or m are 1;
   the symbols [symbol] and [symbol] independently represent a single or a double bond, and
   if the symbol [symbol] is a double bond, p and v are 0 and m is 1 or p and m are 0 and v is 1;
   if the symbol [symbol] is a double bond q and r are 0 and u is 1 or q and u are 0 and r is 1;
   if the symbol [symbol] is a single bond p, v and m are 1;
   if the symbol [symbol] is a single bond q, r and u are 1; and
   when the compound of formula (I) is cyclic:
   the symbols [symbol] and [symbol] are a double bond, and m, v, p, q, n and u are 0 and r is 1, or m, v, p, q, n and r are 0 and u is 1;
said process comprising the steps of:
   a) providing one or more elastomers, a compound of formula (I), and at least one additional reinforcing filler; and
   b) performing at least one mixing step.

Preferably, said Y is O.

Preferably, said R₁ and R₆ are independently selected from the group consisting of hydrogen, linear or branched C₁-C₆ alkyl, linear or branched C₂-C₆ alkenyl, aryl.

Preferably, said R₂-R₅ are independently selected from the group consisting of hydrogen, C₂-C₆ acyl, C₁-C₆ alkyl, linear or branched C₂-C₆ alkenyl, aryl.

Preferably, in the compounds of formula (I) said Y and X₁ are O; said R₁ and R₆ are independently selected from the group consisting of hydrogen, linear or branched C₁-C₆ alkyl, linear or branched C₂-C₆ alkenyl, and aryl; and said R₂-R₅ are independently selected from the group consisting of hydrogen, C₂-C₆ acyl, C₁-C₆ alkyl, linear or branched C₂-C₆ alkenyl, and aryl.

In an embodiment of the present invention said compound of formula (I) is a compound represented by formula (II):

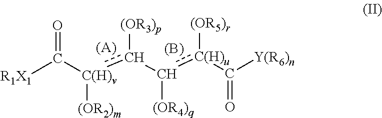

wherein n is 1; and wherein the symbols [symbol] and [symbol] independently represent a single or a double bond, and
if the symbol [symbol] is a double bond, p and v are 0 and m is 1 or p and m are 0 and v is 1;
if the symbol [symbol] is a double bond q and r are 0 and u is 1 or q and u are 0 and r is 1;
if the symbol [symbol] is a single bond p, v and m are 1;
if the symbol [symbol] is a single bond q, r and u are 1
and wherein X₁, Y, and R₁-R₆, when present, are as defined above.

Preferably, said compound of formula (II) is a compound represented by one of the following formulae (III)-(VII):

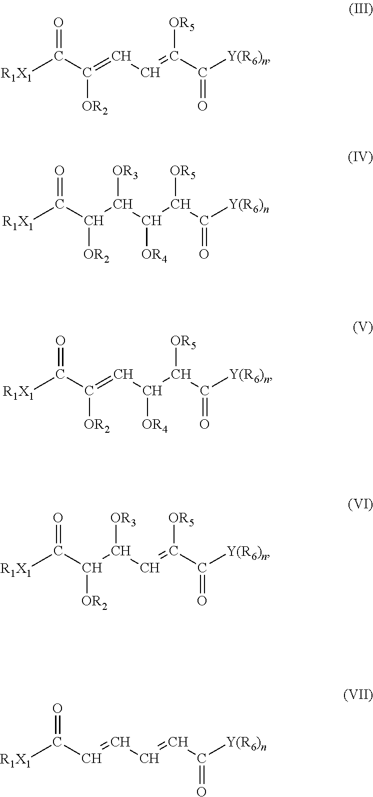

wherein n is 1; and X₁, Y and R₁-R₆, when present, are as defined above.

In alternative embodiments of the present invention, said compound of formula (I) is a compound represented by formula (VIII):

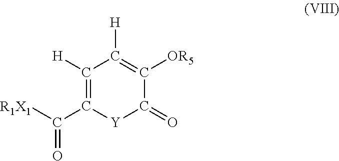

wherein Y, X₁, R₁, and R₅ are as defined above.

Preferably, in the compounds of formulae (II)-(VIII), when present, said Y and X₁ are O; said R₁ and R₆ are independently selected from the group consisting of hydrogen, linear or branched C₁-C₆ alkyl, linear or branched C₂-C₆ alkenyl, and aryl; and said R₂-R₅ are independently selected from the group consisting of hydrogen, C₂-C₆ acyl, C₁-C₆ alkyl, linear or branched C₂-C₆ alkenyl, and aryl.

In particularly preferred embodiments of the present invention, said compound of formulae (I)-(VIII) is selected from the group consisting of:

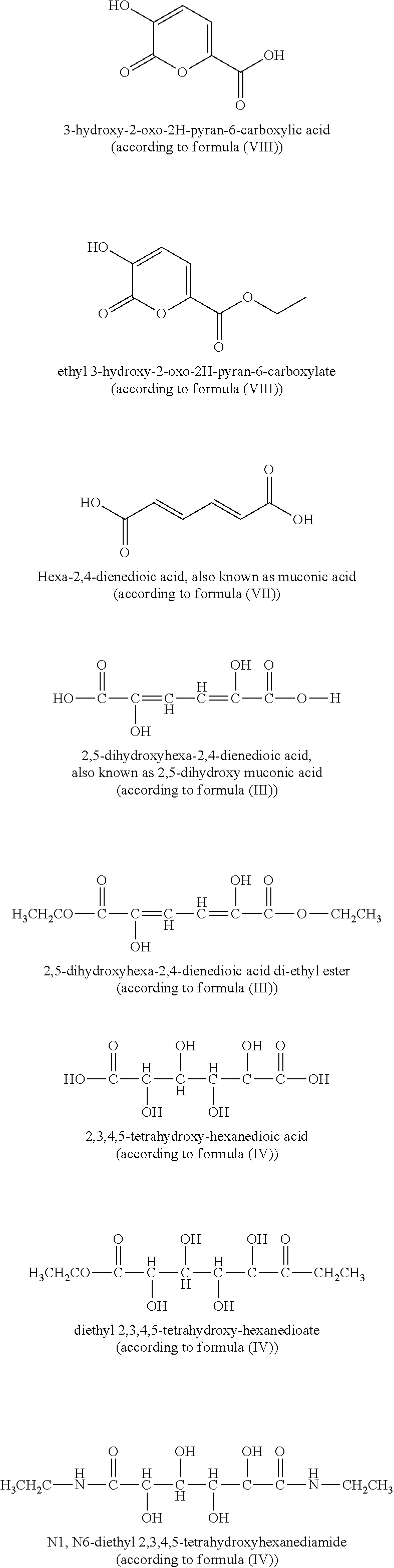

Preferably, said 2,5-dihydroxy muconic acid is a mixture of trans-trans, cis-trans, and cis-cis isomers.

Typically said compound of formula (I) is provided in an amount of at least 0.1 phr, preferably of from 0.5 phr to 6 phr, even more preferably of from 0.5 phr to 4 phr, for example of from 0.5 phr to 2 phr, or of from 0.99 phr to 2 phr.

Typically, at least one of said one or more elastomers provided in step a) of the process according to the present invention is an unsaturated elastomer which is preferably selected in the group consisting of: poly(1,4-cis-isoprene), either natural rubber or synthetic polymer, poly(3,4-isoprene), poly(butadiene) (in particular poly(butadiene) with a high content of 1,4-cis units, i.e. poly(1,4-cis-butadiene)), isoprene/isobutene copolymers, halogenated isoprene/isobutene copolymers such as for example halogenated butyl rubber, in particular chlorobutyl and bromobutyl rubber, 1,3-butadiene/acrylonitrile copolymers, styrene/1,3-butadiene copolymers, styrene/isoprene/1,3-butadiene copolymers, styrene/1,3-butadiene/acrylonitrile copolymers, and mixtures thereof.

Advantageously, at least one elastomer of one or more mono-olefins can be further provided. The mono-olefins can be selected from: ethylene and 1-olefins with 3 to 12 carbon atoms, such as, for example, propylene, 1-butene, 1-pentene, 1-hexene, 1-octene, or mixtures of these mono-olefins.

The elastomer of one or more mono-olefins may comprise a diene, which generally has from 4 to 20 carbon atoms and is preferably selected from: 1,3-butadiene, isoprene, 1,4-hexadiene, 1,4-cyclohexadiene, 5-ethylidene-2-norbornene, 5-methylene-2-norbornene, vinylnorbornene or mixtures of these dienes. The diene can optionally be halogenated.

Among these elastomers of one or more mono-olefins, the following are preferred: ethylene/propylene copolymers (EPR) or ethylene/propylene/diene copolymers (EPDM) and poly(isobutene).

Advantageously, in step a) of the process according to the present invention it can be further provided an unsaturated, diene or non-diene monomers-based elastomer, functionalized by reaction with a suitable terminating agent or coupling agent. In particular, the diene elastomer polymer can be obtained by anionic polymerization promoted by an organometallic initiator (in particular an alkyl-lithium) and terminated by reaction with suitable terminating agents or coupling agents such as, for example, epoxides, carbonyl compounds such as for example cyclohexanone and benzophenone, substituted or unsubstituted, imines, carbodiimides, alkyl-tin halides, alkoxysilanes or aryloxysilanes.

Preferably, said at least one reinforcing filler is selected from the group comprising: sp² hybridized carbon allotropes, silica, layered silicates, mixed oxides of aluminium and magnesium with lamellar structure, alumina and silico aluminates and mixtures thereof.

Preferably said sp² hybridized carbon allotropes are selected from the group consisting of: carbon black, graphene, 2 layer graphene, from 3 to 10 layer graphene, graphite, high surface area graphite, single wall or multiwall carbon nanotubes, carbon nanotubes of longitudinal or helicoid extension, nanocones, nanohorns, nanotoroids, fullerene and mixtures thereof.

Even more preferably, said sp² hybridized carbon allotropes are selected from the group consisting of: carbon black, graphene, graphite, high surface area graphite, single wall or multiwall carbon nanotubes and mixtures thereof.

In a particularly preferred embodiment, said sp² hybridized carbon allotrope is carbon black.

Typically, said sp² hybridized carbon allotrope is provided in an amount of at least 1 phr, preferably of between 2 phr and 70 phr, even more preferably of between 3 phr and 50 phr, for example from 10 phr to 50 phr, or from 15 phr to 50 phr.

In embodiments of the present invention, the at least one reinforcing filler is selected from carbon black or a mixture of carbon black and silica. In such embodiments, carbon black is provided in an amount of at least 10 phr, preferably of at least 15 phr, even more preferably of from 30 phr to 70 phr.

In embodiments of the invention silica is provided in an amount of from 0 to 100 phr, preferably of from 0 to 50 phr, even more preferably of from 0 phr to 25 phr.

Therefore in the process according to the present invention, the total amount of reinforcing fillers provided may reach from 10 to 150 phr, preferably from 20 to 150 phr, even more preferably from 30 to 80 phr.

In embodiments of the present invention, one or more additives can be further provided in step a). The additives are selected on the basis of the specific application for which the composition is intended, and can for example be anti-ageing agents, such as antioxidants and/or anti-ozone agents, plasticizers, adhesives, modifying resins, coupling agents, or mixtures thereof.

In an embodiment of the invention, in step a) there is provided at least one anti-ageing agent selected from antioxidants, anti-ozone agents, and mixtures thereof.

In a particularly preferred embodiment, in step a) there is provided at least one antioxidant or at least one anti-ozone agent.

Typically, said antioxidant and said anti-ozone agent are each provided, either alone or in combination between each other, independently in an amount of 0.1 phr to 20 phr, preferably from 0.5 phr to 20 phr.

A preferred anti-ozone agent is 1,3-dimethylbutyl)-N′-phenyl-p-phenylenediamine (6PPD).

A preferred antioxidant agent is 2,2,4-trimethyl-1,2-dihydroquinoline (TMQ).

In particular, for the purpose of improving workability, a plasticizer generally selected from mineral oils, plant oils, synthetic oils or mixtures thereof, such as, for example, aromatic oil, naphthenic oil, phthalates, soya oil or mixtures thereof can be added to said elastomer composition. The amount of plasticizer is generally from 0 phr to about 70 phr, preferably from about 5 phr to about 30 phr.

In embodiments of the present invention, one or more vulcanizing agents can be further provided. Said vulcanizing agents comprise vulcanization systems based on sulphur, comprising sulphur or molecules comprising sulphur (sulphur donors), together with vulcanization accelerators and/or activators known in the art.

The activators which are particularly effective are compounds of zinc, in particular ZnO, ZnCO₃ and zinc salts of saturated or unsaturated fatty acids comprising from 8 to 18 carbon atoms, such as, for example, zinc stearate, which are preferably formed in situ in the elastomer composition from ZnO and fatty acid, or mixtures thereof.

The accelerators which are commonly used can be selected from: dithiocarbamates, guanidine, thiourea, thiazoles, sulfenamides, thiurams, amines and xanthates and mixtures thereof.

Preferred vulcanising agents are for example, stearic acid, ZnO, TBBS (N-tert-butyl-2-benzothiazyl sulfenamide) and sulphur or mixtures thereof.

According to a preferred embodiment, said vulcanizing agent is provided in an amount greater than or equal to about 1 phr, preferably greater than or equal to about 2 phr.

Preferably, the quantity of vulcanizing agent is less than or equal to about 20 phr, preferably less than or equal to about 10.

Advantageously the quantity of sulphur lies between about 0.5 phr and about 8 phr.

Typically, said at least one mixing step is performed by mixing together the polymeric components with the compound of formula (I) according to the present invention, and together with the reinforcing fillers and the other additives possibly present, according to techniques known in the art.

The mixing can be performed, for example, using an open mixer of the “open-mill” type and/or an internal mixer of the type with tangential rotors (Banbury®), and/or with intermeshing rotors (Intermix™), and/or in continuous mixers of the Ko-Kneader™ type, and/or of the twin screw or multiscrew type and/or of the planetary type.

The ingredients are not generally introduced all simultaneously into the mixer but are typically added in sequence. In particular, the vulcanization additives, such as the vulcanizing agents including possibly an activator and/or an accelerator, are usually added in a downstream step relative to the incorporation and processing of all the other components.

Typically, this is a further mixing step which takes place according to the known techniques, in particular with vulcanization systems based on sulphur commonly used for diene elastomer polymers. For this purpose, in the materials, after one or more steps of thermal-mechanical treatment, a vulcanizing agent based on sulphur is incorporated together with vulcanization accelerators. At the final treatment step, the temperature is generally maintained below 120° C. and preferably below 100° C., so as to avoid any undesired pre-crosslinking phenomenon.

Therefore, in an embodiment the process according to the present invention further comprises the following steps of:
c) providing one or more vulcanizing agents, optionally comprising an activator and/or an accelerator, as disclosed above; and
d) performing a further mixing step.

The mixing steps of the process according to the present invention can be performed at a temperature of from 40° C. to 130° C.

In the vulcanizable elastomer composition, the individual components of the elastomer composition do not always remain unaltered or are individually traceable in that they may be transformed, completely or in part, by effect of the interaction with other components, or by effect of the energy such as heat and/or the mechanical energy provided.

### Experimental Part

### Materials

All the reagents and solvents for the preparation of the pyrone derivatives, as well as adipic acid and muconic acid, were obtained from Sigma-Aldrich and used without further purification.

The ¹H-NMR spectra were recorded on samples dissolved in deuterated chloroform (CDCl₃) and dimethyl sulfoxide (DMSO-d₆).

The elastomers used are:
poly(1,4-cis-isoprene) (NR), solid natural rubber SIR20, from Eatern GR Thailand—Chonburi. Mooney viscosity (ML(1+4) @ 100° C.): 73 MU;
poly(1,4-cis-butadiene) (butadiene rubber, BR) Europrene Neocis® from Polimeri Europa;
IR (isoprene rubber) SKI-3 from Nizhnekamskneftekhim;

The sp² hybridized carbon allotrope used is:
Carbon black N326 from Birla Carbon;
Carbon black N550 from Birla Carbon

Other ingredients for the preparation of the elastomer compositions:
X50S (bis(triethoxysilylpropyl) polysulfide), from Evonik;
Silica from grade Zeosil® 1165 MP from Solvay;
Stearic acid from Undesa;
ZnO from Zincol Ossidi;
6PPD ((1,3-dimethylbutyl)-N′-phenyl-p-phenylenediamine) Santoflex™ from Eastman;
TBBS (N-tert-butyl-2-benzothiazyl sulphenamide) Vulkacit® NZ/EGC—from Lanxess;
Sulphur from International Sulphur Inc;
TMQ (2,2,4-Trimethyl-1,2-Dihydroquinoline) NAUGARD Q from CHEMTURA CORPORATION;
Resin: Escorez 1102 from Exxon mobilWax: Riowax from SER corporation.

### Characterization

The pyrone derivatives obtained were analysed by NMR spectroscopy. The ¹H-NMR and ¹³C-NMR spectra were recorded with a Bruker 400 MHz (100 MHz ¹³C) instrument operating at 298 K. The chemical shifts are stated in parts per million (ppm) with the solvent residues peaks as internal standard (DMSO-d₆: δ=2.50 ppm, CDCl₃: δ=7.26 ppm).

Thermogravimetric analysis (TGA) is a quantitative analytical technique which provides the weight loss of the material analysed as a function of time and temperature. In other words, the material is heated with a given heating regime and can undergo transformations which lead to the loss of part or all of the material itself, which passes into the vapour phase. The TGA under flowing air (60 mL/min) was performed using a Mettler TGA SDTA/851 instrument. The samples (10 mg) were heated from 25 to 400° C. at 10° C./min, maintaining at 400° C. for 20 min.

Differential Scanning Calorimetry (DSC) was performed under flowing oxygen (70 mL/min) to measure Oxidation Onset Temperature. During the tests, 3±5 mg of sample was placed into an aluminium pan and heated from 20° C. to 380° C. with a constant heating rate of 10 C.°/min in an oxygen flow of 70 ml/min. The extrapolated temperatures of the start of the oxidation and temperatures of the maximum heat flow were determined from each DSC scan by using program STAR (TA Instruments). Oxidation tests were performed on elastomer compositions samples in order to compare their thermal stability by means of Oxidation Onset Temperatures (OOT). The Oxidation Onset Temperature was obtained in non-isothermal (dynamic) condition, from the intersection of the extrapolated baseline and the tangent line of the exothermic peak using a thermal analysis software (STAR). All experiments were carried out in duplicate. Similar conditions were used for the isothermal oxidation test.

Ozone dynamic test was determined according to ISO 1431-1 and using air containing 50 pphm of ozone, for 70 h at 50° C., with 20% of stress at 0.5 Hz. The test pieces were visually examined for cracks.

Tensile measurements in accordance with the standard ISO 37:2005 were performed on samples of the elastomer compositions vulcanized at 175° C. for 20 minutes. In particular, the load was measured at different levels of elongation, 50%, 100% and 300%, named in sequence σ₅₀, σ₁₀₀, σ₃₀₀, and the stress at break and the elongation at break named in sequence σ_{B} and ε_{B}. The tensile tests were performed on test pieces of the dumbbell type with rectilinear axis.

The dynamic mechanical properties were measured using an Instron dynamic device in the traction-compression mode according to the following methods. A test piece of the crosslinked elastomer composition having a cylindrical form (length=25 mm; diameter=12 mm) and kept at the prefixed temperature (10, 23 and 70° C.) for the whole duration of the test, was compression-preloaded up to a 25% longitudinal deformation with respect to the initial length and then submitted to a dynamic sinusoidal strain having an amplitude of ±3.5% with respect to the length under pre-load, with a 100 Hz frequency. The dynamic-mechanical properties are expressed in terms of dynamic storage modulus (E′) and loss factor (Tan delta) values. The tan delta value is computed as the ratio between the loss (E″) and the storage (E′) modulus.

The static and dynamic mechanical properties measured are summarized in the following table 1.

TABLE 1

| | |
|---|---|
| Symbol | Meaning |

Static mechanical properties

| | |
|---|---|
| σ₅₀ | Stress at 50% elongation |
| σ₁₀₀ | Stress at 100% elongation |
| σ₃₀₀ | Stress at 300% elongation |
| σ_{B} | Stress at break |
| ε_{B} | Elongation at break |

Vulcanization properties

| MH | maximum torque |
|---|---|
| ML | minimum torque |
| ts1 | time needed to have an increase of 1 DNm of the |
| | torque. Index of scorch time |
| t90 | time needed to achieve 90% of maximum modulus. |
| | Index of optimal vulcanization time. |
| Dynamic mechanical properties measured by means of | |
| axial stress | |
| E′ | Storage modulus |
| E″ | Loss (viscous) modulus |
| tan delta | E″/E′ |

### Synthesis of Pyrone Derivatives

### Example 1. Synthesis of 3,4,5-triacetoxy-6-oxo-tetrahydro-pyran-2-carboxylic Acid (1) (Precursor)

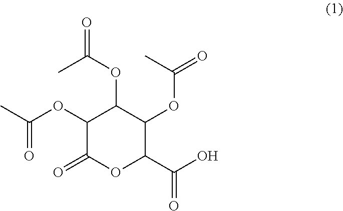 [SMILES: CC(=O)OC1C(=O)OC(C(=O)O)C(OC(C)=O)C1OC(C)=O]

A 100-mL flask equipped with a magnetic stirrer and a condenser was charged with 10 g of mucic acid (0.049 mol) and 51 mL of acetic anhydride (0.54 mol). The mixture was stirred at 130° C. overnight. The pure product was isolated by filtration (yield 99%).

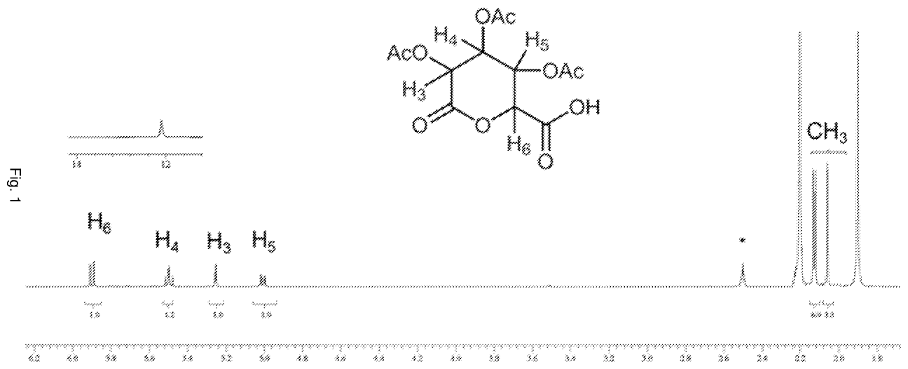

The product was characterized by NMR spectroscopy and the ¹H NMR spectrum is showed in FIG. 1 (400 MHz, DMSO-d6, δ in ppm): 2.08 (s, 3H); 2.15 (s, 6H); 5.01 (dd, 1H); 5.27 (d, 1H); 5.51 (t, 1H); 5.89 (d, 1H); 12.10 (s, 1H).

### Example 2. Synthesis of 3-hydroxy-2-oxo-2H-pyran-6-carboxylic Acid (Compound 2)

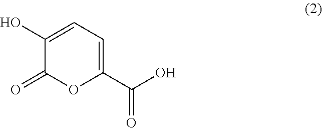 [SMILES: O=C(O)c1ccc(O)c(=O)o1]

A 100-mL flask equipped with a magnetic stirrer and a condenser was charged with 1 g of CH₃COONa·3H₂O (0.049 mol) and the reaction mixture obtained in example 1. The new reaction mixture was then stirred for 12 hours at 100° C. At the end of this time, HCl solution (0.0002 mol; 2 ml; 36%) was added and a few minutes after addition there was formation of a white precipitate (59.8 g). Once formed, the precipitate was removed by filtration. The solution, on the other hand, was concentrated at reduced pressure until about 32 g of solution is obtained. The solution thus obtained was left to stand until a new precipitate had formed. After 1 day, the procedure was repeated and a further 3.2 g of solid was isolated. The solid product of the 3 filtrations was characterized by NMR spectroscopy (yield: 65%).

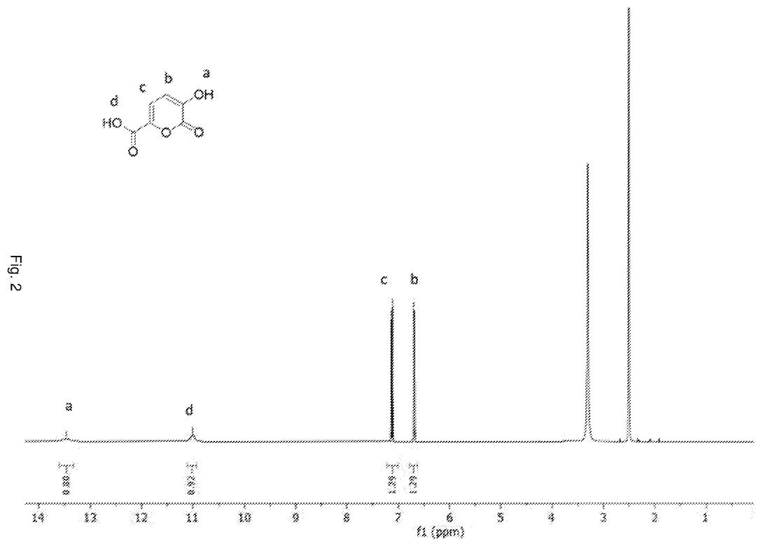

The ¹H NMR spectrum is showed in FIG. 2 (400 MHz, DMSO-d6, δ in ppm): 2.27 (s, 3H); 7.13 (d, 1H); 7.47 (d, 1H); 12.10 (s, 1H).

### Example 3. Synthesis of the ethyl 3-hydroxy-2-oxo-2H-pyran-6-carboxylate (Compound 3)

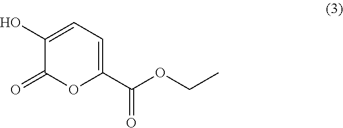

A 100-mL flask equipped with a magnetic stirrer and a condenser was charged with the product obtained in example 2 (compound (2)) (3.1 g), ethanol (30 ml) and six drops of sulfuric acid (1.0 g; 0.049 mol). The mixture thus obtained was stirred at 85° C. overnight. At the end of this time, the reaction mixture was poured into a separatory funnel and water was added (180 ml) and the pH was neutralized by adding a small amount of sodium bicarbonate. The mixture was extracted 3 times with dichloromethane (60 ml). The residual water was brought to a basic pH with sodium bicarbonate. The organic phases were dried over sodium sulphate and then filtered and dried thoroughly at reduced pressure (yield: 99%).

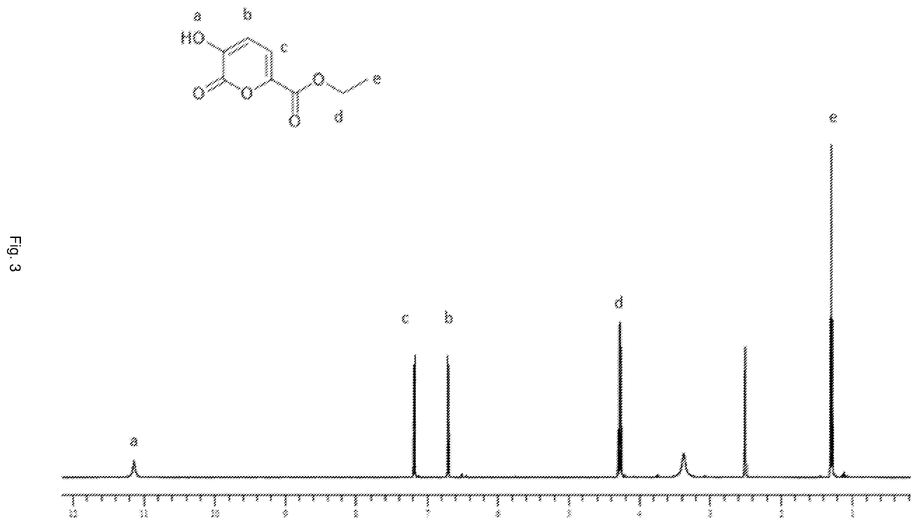

The product was characterized by ¹H NMR spectroscopy and the spectrum is showed in FIG. 3 (400 MHz, CDCl₃-d6, δ in ppm): 1.40 (t, 3H); 4.40 (q, 2H); 6.78 (d, 1H); 7.20 (d, 1H); 13C NMR (100 MHz, CDCl3): δ 14.17, 62.17, 112.93, 113.00, 140.85, 145.70, 159.14, 159.59 ppm).

### Examples 4-8: Partial Replacement of Silica in Elastomer Compositions

Elastomer compositions 4-8 were prepared via melt blending, according to the quali-quantitative composition summarized in Table 2. Reference composition 4\hat{ } comprised silica as main reinforcing filler, together with carbon black; in the compositions 5-7 silica has been partially replaced by a compound according to the present invention and an additional amount of carbon black, whilst in reference composition 8\hat{ } the same has been done using adipic acid, the saturated analogous of muconic acid.

TABLE 2

| | Ex. 4\hat{ } | Ex. 5 | Ex. 6 | Ex. 7 | Ex. 8\hat{ } |
|---|---|---|---|---|---|
| | [phr] | [phr] | [phr] | [phr] | [phr] |
| NR (SIR-20) | 70 | 70 | 70 | 70 | 70 |
| BR | 30 | 30 | 30 | 30 | 30 |
| CB N326 | 30 | 30 | 30 | 30 | 30 |
| SILICA | 35 | 12 | 12 | 12 | 12 |
| X50S | 5.6 | 5.6 | 5.6 | 5.6 | 5.6 |
| ZnO | 4 | 4 | 4 | 4 | 4 |
| Stearic acid | 2 | 2 | 2 | 2 | 2 |
| 6PPD | 2 | 2 | 2 | 2 | 2 |
| TBBS | 1.8 | 1.8 | 1.8 | 1.8 | 1.8 |
| Sulphur | 2 | 2 | 2 | 2 | 2 |
| CB N326 | 0 | 18.73 | 18.73 | 18.73 | 18.73 |
| Compound (2) | 0 | 0.99 | 0 | 0 | 0 |
| Compound (3) | 0 | 0 | 0.99 | 0 | 0 |
| Muconic acid | 0 | 0 | 0 | 0.99 | 0 |
| Adipic acid | 0 | 0 | 0 | 0 | 0.99 |

\hat{ }comparison

### Example 4—Elastomer Composition Comprising Silica and CB (Comparative Example)

70 phr of Natural Rubber (SIR-20) and 30 phr of poly(1,4-butadiene) (BR) were fed into a Brabender® internal mixer and masticated at 130° C. for 1 minute. CB, Silica Zeosil 1156MP and X50S were added into the mixer and mixed for 3 minutes at the same temperature. ZnO, stearic acid and 6PPD were then added and mixed for 2 minutes.

The so obtained compound was unloaded at 130° C.

These composites were fed again into the internal mixer at 45° C. Sulphur and TBBS (N-tert-butyl-2-benzothiazyl sulfenamide) were then added, mixing for a further 2 minutes.

The final rubber composite was discharged and cooled at room temperature and, before the characterization, mixed few times in 2 rolls open mill.

### Example 5—Elastomer Composition Comprising Silica, CB, and Compound 2

The procedure disclosed in Example 4 was repeated, however 66% of the silica was replaced with pristine CB and 0.99 phr of compound (2), obtained as disclosed in Example 2. The same volume % of the filler was maintained.

### Example 6—Elastomer Composition Comprising Silica, CB, and Compound 3

The procedure disclosed in Example 4 was repeated, however 66% of the silica was replaced with pristine CB and 0.99 phr of compound (3), obtained as disclosed in Example 3. The same volume % of the filler was maintained.

### Example 7—Elastomer Composition Comprising Silica, CB, Muconic Acid

The procedure disclosed in Example 4 was repeated, however 66% of the silica was replaced with pristine CB and 0.99 phr of muconic acid. The same volume % of the filler was maintained.

### Example 8—Elastomer Composition Comprising Silica, CB, and Adipic Acid (Comparative Example)

The procedure disclosed in Example 4 was repeated, however 66% of the silica was replaced with pristine CB and 0.99 phr of adipic acid. The same volume % of the filler was maintained.

### Dynamic Mechanical Characterization of Elastomer Compositions 4-8 by Means of Axial Stress

In Table 3, the data obtained from the dynamic mechanical tests, performed by application of a sinusoidal axial stress, are shown. The experimental conditions for performing the tests have been described above.

TABLE 3

| | Ex. 4\hat{ } | Ex. 5 | Ex. 6 | Ex. 7 | Ex. 8\hat{ } |
|---|---|---|---|---|---|
| E′@10° C. | 3.88 | 3.74 | 3.84 | 3.81 | 3.91 |
| E″@10° C. | 0.66 | 0.68 | 0.70 | 0.68 | 0.78 |
| TanDelta@10° C. | 0.170 | 0.181 | 0.181 | 0.178 | 0.200 |
| E′@23° C. | 3.69 | 3.51 | 3.60 | 3.57 | 3.47 |
| E″@23° C. | 0.55 | 0.57 | 0.59 | 0.57 | 0.64 |
| TanDelta@23° C. | 0.149 | 0.163 | 0.163 | 0.160 | 0.184 |
| E′@70° C. | 3.32 | 3.16 | 3.24 | 3.22 | 2.95 |
| E″@70° C. | 0.46 | 0.50 | 0.51 | 0.50 | 0.60 |
| TanDelta@70° C. | 0.139 | 0.159 | 0.158 | 0.155 | 0.203 |
| ΔE′ (E_{10° C.}′- | 0.56 | 0.58 | 0.60 | 0.59 | 0.96 |
| E_{70° C.}′) | | | | | |

\hat{ }comparison

The use of the compounds according to the present invention (compounds 2, 3, and muconic acid, respectively Ex. 5, 6 and 7) as ingredients of the elastomer composition, allows the partial replacement of silica with CB, substantially reproducing the same dynamic-mechanical properties measured for the reference composition (ex. 4\hat{ }). This behaviour was not observed for the mixture with adipic acid, which does not contain unsaturations nor —OH groups in alfa position to the carbonyl.

Besides this general comment, it is important to observe that:
(i) the values of the storage modulus E′ are slightly lower at low temperature for the elastomer compositions of the invention (Ex. 5-7), with respect to the reference composition (Ex. 4\hat{ }). This means that the composition has lower rigidity at low temperature, and this is definitely an advantage;
(ii) The values of ΔE′ (E′10° C.-E′70° C.) are substantially in line (slightly higher) for the compositions of the invention (Ex. 5-7), with respect to the reference composition (Ex. 4\hat{ }). This means that, although silica was partially replaced with CB, the compositions of the invention have substantially the same stability of the dynamic rigidity as the temperature increases with respect to the reference composition;
(iii) the composition comprising adipic acid (Ex. 8\hat{ }) shows a value of E′ at low temperature slightly higher and, more than that, the value of ΔE′ (E′10° C.-E′70° C.) is much larger than the values of the reference composition (Ex. 4\hat{ }) and of the invention compositions (Ex. 5-7);
(iv) the values of tan delta are substantially in line at all the temperatures for the compositions of the invention (Ex. 5-7), with respect to the reference composition (Ex. 4\hat{ }), whilst they are appreciably higher for the composition comprising adipic acid (Ex. 8\hat{ }). It is known that it is beneficial for a rubber composition for tyre tread to have higher hysteresis at low temperature and lower tan delta at high temperature. Instead, for an elastomeric composite for other tyre compositions, it is beneficial to have a low hysteresis at every temperature.

### Characterization of the Elastomer Compositions 4-8 by Means of Tensile Tests

Tensile properties were determined by quasi static measurements as disclosed above. In Table 4, the data obtained from the tensile tests are shown.

TABLE 4

| | Ex. 4\hat{ } | Ex. 5 | Ex. 6 | Ex. 7 | Ex. 8\hat{ } |
|---|---|---|---|---|---|
| σ₅₀ (Mpa) | 0.76 | 0.77 | 0.74 | 0.81 | 0.60 |
| σ₁₀₀ (MPa) | 1.08 | 1.09 | 1.03 | 1.18 | 0.78 |
| σ₃₀₀ (MPa) | 4.09 | 4.05 | 3.68 | 4.60 | 2.02 |
| σ_{B} (MPa) | 22.36 | 23.92 | 21.38 | 19.90 | 9.43 |
| ε_{B} (%) | 692.01 | 749.08 | 713.94 | 653.98 | 667.22 |
| Energy J/cm³ | 52.80 | 62.78 | 52.06 | 46.39 | 22.87 |

\hat{ }comparison

The tensile properties confirm what already reported commenting the data from dynamic-mechanical characterization.

Indeed, the elastomer compositions of the invention (Ex. 5-7) have tensile properties which are in line with those of the reference composition (Ex. 4\hat{ }). In particular, it is worth observing that the composition comprising compound 3 (Ex. 7) has better overall ultimate properties.

On the contrary, the composition comprising adipic acid (Ex. 8\hat{ }) revealed appreciably different values of the tensile properties: much lower values of stresses at all the elongation and much worse overall ultimate properties. It appears that adipic acid does not promote the reinforcement of the elastomeric composite.

### Examples 9-16: Compounds of the Invention as Antioxidant and Anti-Ozone Agents in Elastomer Compositions

Elastomer compositions 9-16 were prepared via melt blending, according to the quali-quantitative composition summarized in Table 5 below.

Briefly, 50 phr of Isoprene Rubber (IR) and 50 phr of poly(1,4-butadiene) (BR) were fed into a Brabender® internal mixer and masticated at 110° C. for 1 minute. CB was added into the mixer and mixed for 3 minutes at the same temperature. ZnO, stearic acid, wax, resin and the selective protective agent(s) (TMQ, 6PPD, or compound 3) were then added and mixed for 2 minutes. The so obtained compound was unloaded at 110° C.

These composites were fed again into the internal mixer at 80° C. Sulphur and TBBS (N-tert-butyl-2-benzothiazyl sulfenamide) were then added, mixing for a further 2 minutes. The final rubber composite was discharged and cooled at room temperature and, before the characterization, mixed few times in 2 rolls open mill.

TABLE 5

| | Ex. | Ex. | Ex. | Ex. | Ex. | Ex. | Ex. | Ex. |
|---|---|---|---|---|---|---|---|---|
| | 9\hat{ } | 10\hat{ } | 11\hat{ } | 12\hat{ } | 13\hat{ } | 14\hat{ } | 15 | 16 |
| | [phr] | [phr] | [phr] | [phr] | [phr] | [phr] | [phr] | [phr] |
| IR | 50 | 50 | 50 | 50 | 50 | 50 | 50 | 50 |
| BR | 50 | 50 | 50 | 50 | 50 | 50 | 50 | 50 |
| CB N550 | 45 | 45 | 45 | 45 | 45 | 45 | 45 | 45 |
| Stearic acid | 2 | 2 | 2 | 2 | 2 | 2 | 2 | 2 |
| ZnO | 2.85 | 2.85 | 2.85 | 2.85 | 2.85 | 2.85 | 2.85 | 2.85 |
| Wax | 2 | 2 | 2 | 2 | 2 | 2 | 2 | 2 |
| Resin | 2 | 2 | 2 | 2 | 2 | 2 | 2 | 2 |
| TBBS | 0.9 | 0.9 | 0.9 | 0.9 | 0.9 | 0.9 | 0.9 | 0.9 |
| Sulphur | 3 | 3 | 3 | 3 | 3 | 3 | 3 | 3 |
| TMQ | 0 | 4 | 0 | 2 | 0 | 2 | 0 | 2 |
| 6PPD | 0 | 0 | 4 | 0 | 2 | 2 | 2 | 0 |
| Compound (3) | 0 | 0 | 0 | 0 | 0 | 0 | 2 | 2 |

\hat{ }comparison

Elastomer compositions 9-16 above all comprise carbon black (CB N550) as the filler. Reference composition 9\hat{ } does not comprise any antioxidant/anti-ozone system; reference compositions 10\hat{ } and 12\hat{ } comprise only TMQ; reference compositions 11\hat{ } and 13\hat{ } comprise only 6PPD; reference composition 14\hat{ } comprises both TMQ and 6PPD. In the compositions according to the present invention a compound according to the invention, in particular a pyrone derivative (compound 3), is used together with either 6PPD (Ex. 15) or TMQ (Ex. 16).

### Evaluation of Oxidation Onset Temperature (OOT) Based on DSC Analysis

OOT tests were performed on elastomer compositions 9-16 disclosed above. Data are summarized in Table 6.

TABLE 6

| | OOT 1 | OOT 2 | OOT 3 |
|---|---|---|---|
| Example | (° C.) | (° C.) | (° C.) |
| 9\hat{ } | 202 | 300 | 380 |
| 10\hat{ } | 238 | 380 | — |
| 11\hat{ } | 255 | 380 | — |
| 12\hat{ } | 225 | 370 | — |
| 13\hat{ } | 249 | 364 | — |
| 14\hat{ } | 251 | 367 | — |
| 15 | 246 | 360 | — |
| 16 | 224 | 369 | — |

\hat{ }comparison

Compound 3 appears to be a suitable antioxidant and anti-ozone ingredient in an elastomer composition, in particular when it is used in replacement of only one of the two oil-based substances, that means either in replacement of 6PPD (Ex. 16) or in replacement of TMQ (Ex. 15).

By comparing Ex. 14\hat{ } and Ex. 15 it can be seen that the same values of OOT 1 and OOT 2 were obtained. Indeed, the replacement of TMQ with compound 3 results in values substantially in line with the reference compound. A higher value derived from exothermic peak integration is recorded.

On the other hand, by comparing Ex. 14\hat{ } and Ex. 16, it can be seen that the replacement of 6PPD with compound 3 results in a significant reduction of OOT value.

### Evaluation of Thermal Stability Based on Thermogravimetric Analysis

The two elastomer compositions that showed the most promising results in the previous test, i.e. the compositions of Ex. 15-16, were analysed via TGA and compared to reference compositions 9\hat{ }, comprising no antioxidant/anti-ozone system, and 14\hat{ }, comprising both TMQ and 6PPD. Data from TGA analysis are summarized in Table 7.

TABLE 7

| | T < | T > | Final | % @45 |
|---|---|---|---|---|
| Example | 300° C. | 300° C. | Residue | min |
| 9\hat{ } | ND | 40.0 | 59% | 67% |
| 14\hat{ } | 3.77 | 34.8 | 61% | 70.5% |
| 15 | 5.28 | 31.0 | 63.58% | 71% |
| 16 | 3.79 | 30.1 | 66% | 73% |

\hat{ }comparison

TGA analysis shows that the substitution of 6PPD or TMQ with a pyrone derivative (i.e. compound 3) leads to greater thermal stability, indicated by the amount of residual mass, compared to the reference composition (Ex. 14\hat{ }).

### Vulcanization of Elastomer Compositions 14-16

Elastomer compositions of Ex. 15-16 and reference composition 14\hat{ } were vulcanized at 170° C. for 10 minutes. The vulcanization properties measured are summarized in the following table 8, wherein the curing rate is defined through the following formula:

$$Curing rate = {M}_{H} - {M}_{L} / {t}_{90} - {t}_{S 1}$$

TABLE 8

| | Ex. | Ex. | Ex. |
|---|---|---|---|
| | 14\hat{ } | 15 | 16 |
| M_{H} [dNm] | 16.13 | 14.29 | 14.98 |
| M_{L} [dNm] | 1.62 | 1.78 | 1.84 |
| t_{S1} [min] | 2.48 | 2.59 | 2.84 |
| t₉₀ [min] | 4.64 | 4.95 | 5.08 |
| Curing rate | 6.72 | 5.30 | 5.90 |
| [dNm/min] | | | |

\hat{ }comparison

The replacement of either TMQ or 6PPD with a pyrone derivative (i.e. compound 3) led to the increase of ML, which is an index of the composition's viscosity. Moreover, the presence of the pyrone derivative does not affect the scorch time of vulcanization, as revealed by tₛ₁.

### Tensile Properties of Elastomer Compositions 14-16

Tensile properties were determined by quasi static measurements. Data are in Table 9.

TABLE 9

| | Ex. 14\hat{ } | Ex. 15 | Ex. 16 |
|---|---|---|---|
| σ₅₀ (Mpa) | 1.37 | 1.28 | 1.32 |
| σ₁₀₀ (MPa) | 2.44 | 2.14 | 2.22 |
| σ₃₀₀ (MPa) | 10.92 | 8.99 | 9.36 |
| σ_{B} (MPa) | 19.15 | 18.73 | 18.68 |
| ε_{B} (%) | 475.36 | 534.22 | 511.36 |
| Energy J/cm³ | 40.83 | 44.82 | 42.38 |

\hat{ }comparison

The tensile properties of the elastomer composition obtained by replacing TMQ with a pyrone derivative (Ex. 15) appear substantially in line with those of the reference composition (Ex. 14\hat{ }), with better elongation at break and energy at break. The replacement of 6PPD with a pyrone derivative (Ex. 16) does not remarkably affect the tensile properties: stresses are well aligned, and values of the overall ultimate properties are somewhat lower in terms of stress at break, but with higher elongation leading anyway to higher energy at break with respect the reference composition (Ex. 14\hat{ }).

The elastomer compositions were aged for 2 weeks at 70° C. and tensile properties were measured again. This experiment is particularly meaningful as it gives an indication of the ability of the chemical compounds used as antioxidant/anti-ozone agents to protect the elastomer compositions. Data are summarized in Table 10 by reporting the percentage variation of each value when compared with the value measured before ageing.

TABLE 10

| | Ex. 14\hat{ } | Ex. 15 | Ex. 16 |
|---|---|---|---|
| σ₅₀ (Mpa) | 10% | 16% | 11% |
| σ₁₀₀ (MPa) | 14% | 21% | 17% |
| σ₃₀₀ (MPa) | 9% | 17% | 18% |
| σ_{B} (MPa) | −19% | −15% | −11% |
| ε_{B} (%) | −21% | −20% | −18% |
| Energy J/cm³ | −36% | −28% | −26% |

\hat{ }comparison

Very similar results were obtained, thus confirming that pyrone derivatives are suitable chemical compounds for the protection of elastomer compositions from the action of oxygen and ozone. In particular, the ultimate properties are affected less by ageing when in the presence of the pyrone derivative. In particular, ageing affects to a lower extent the composite of example 16, over the whole tested parameters.

### Evaluation of Antiozonant Activity

The elastomeric composition of the Ex. 16, comprising TMQ and compound 3, was analysed trough dynamic ozone test as detailed above, and compared to reference compositions 9\hat{ }, comprising no antioxidant/anti-ozone system, and 14\hat{ }, comprising both TMQ and 6PPD.

Data from Ozone tests are summarized in Table 11.

TABLE 11

| | Ex. 9\hat{ } | Ex. 14\hat{ } | Ex. 16 |
|---|---|---|---|
| OZONE DYNAMIC | 4 | 0 | 0.5 |
| TEST 50 C 70 h | | | |

\hat{ }comparison

A visual inspection was conducted on the samples before and after ozone aging. The evaluation of the samples was indicated by a number in a range from 0 (best evaluation) to 4 (worst evaluation with destruction of the sample due to ozone aging).

It is visible here how both samples of Ex. 14\hat{ } and Ex. 16 are much better than compound of Ex. 9\hat{ } not having protective agents.

Very similar results were obtained with both samples of Ex. 14\hat{ } and Ex. 16 confirming that pyrone derivatives are suitable chemical compounds for the protection of elastomer compositions from ozone.

1-21. (canceled)

22. A process for preparing an elastomer composition comprising a compound represented by formula (I) below: 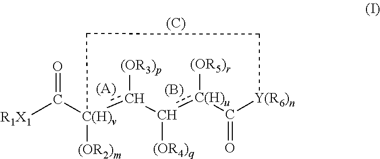
wherein the compound of formula (I) can be linear or cyclic and the symbol [symbol] does not represent a bond when the compound is linear, and it represents a bond when the compound is cyclic; and
m, v, p, q, r, u, and n can independently be either equal to 1 or equal to 0; and X₁ and Y can be O or NR₇;
and wherein
R₇ is selected from: hydrogen, C₂-C₆ acyl, C₁-C₆ alkyl, linear or branched C₂-C₆ alkenyl or alkynyl, aryl, linear or branched C₁-C₆ alkyl aryl, linear or branched C₂-C₆ alkenyl aryl, linear or branched C₂-C₆ alkynyl-aryl, and heteroaryl; and
if X₁ and Y are NR₇ then:
R₁, R₆ are independently selected from: hydrogen, linear or branched C₁-C₆ alkyl, linear or branched C₂-C₆ alkenyl or alkynyl, aryl, linear or branched C₁-C₆ alkyl aryl, linear or branched C₂-C₆ alkenyl aryl, linear or branched C₂-C₆ alkynyl-aryl, and heteroaryl;
if X₁ and Y are O then:
R₁, R₆ are independently selected from: hydrogen, linear or branched C₁-C₆ alkyl, linear or branched C₂-C₆ alkenyl or alkynyl, aryl, linear or branched C₁-C₆ alkyl aryl, linear or branched C₂-C₆ alkenyl aryl, linear or branched C₂-C₆ alkynyl-aryl, heteroaryl, alkali metals, alkaline earth metals, and transition metals, or 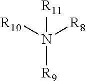
wherein R₈, R₉, R₁₀, R₁₁ are independently selected from the group consisting of: hydrogen, linear or branched C₁-C₆ alkyl, linear or branched C₁-C₆ alkyl aryl, linear or branched C₂-C₆ alkenyl aryl, linear or branched C₂-C₆ alkynyl-aryl, and heteroaryl;
and wherein
R₂, R₃, R₄, R₅ are independently selected from: hydrogen, C₂-C₆ acyl, C₁-C₆ alkyl, linear or branched C₂-C₆ alkenyl or alkynyl, aryl, linear or branched C₁-C₆ alkyl aryl, linear or branched C₂-C₆ alkenyl aryl, linear or branched C₂-C₆ alkynyl-aryl, and heteroaryl;
and wherein:
when the compound of formula (I) is linear:
n is 1 and v, m, or both are 1;
the symbols [symbol] and [symbol] independently represent a single or a double bond, and
if the symbol [symbol] is a double bond, p and v are 0 and m is 1 or p and m are 0 and v is 1;
if the symbol [symbol] is a double bond q and r are 0 and u is 1 or q and u are 0 and r is 1;
if the symbol [symbol] is a single bond p, v and m are 1;
if the symbol [symbol] is a single bond q, r and u are 1; and
when the compound of formula (I) is cyclic:
the symbols [symbol] and [symbol] are a double bond, and m, v, p, q, n and u are 0 and r is 1, or m, v, p, q, n and r are 0 and u is 1;
the process comprising the steps of:
a) providing one or more elastomers, a compound of formula (I), and at least one reinforcing filler; and
b) performing at least one mixing step.

23. The process according to claim 22, wherein the compound of formula (I) is a compound represented by formula (II): 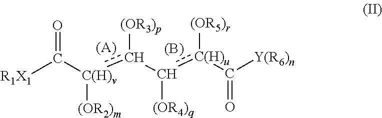
wherein n is 1; and wherein the symbols [symbol] and [symbol] independently represent a single or a double bond, and
if the symbol [symbol] is a double bond, p and v are 0 and m is 1 or p and m are 0 and v is 1;
if the symbol [symbol] is a double bond q and r are 0 and u is 1 or q and u are 0 and r is 1;
if the symbol [symbol] is a single bond p, v and m are 1;
if the symbol [symbol] is a single bond q, r and u are 1
and wherein X₁, Y, and R₁-R₆, when present, are as defined in claim 1.

24. The process according to claim 22, wherein the compound of formula (I) is a compound represented by formula (VIII):

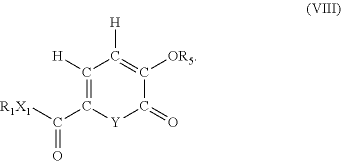

25. The process according to claim 22, wherein the R₁ and R₆ are independently selected from: hydrogen, linear or branched C₁-C₆ alkyl, linear or branched C₂-C₆ alkenyl, and aryl.

26. The process according to claim 22, wherein the R₂-R₅ are independently selected from: hydrogen, C₂-C₆ acyl, C₁-C₆ alkyl, linear or branched C₂-C₆ alkenyl, and aryl.

27. The process according to claim 22, wherein the compound of formula (I) is present in an amount of at least 0.1 phr.

28. The process according to claim 22, wherein at least one of the one or more elastomers is an unsaturated elastomer selected from: poly(1,4-cis-isoprene), natural rubber or synthetic polymer, poly(3,4-isoprene), poly(butadiene), poly(butadiene) with a high content of 1,4-cis units, isoprene/isobutene copolymers, halogenated isoprene/isobutene copolymers, halogenated butyl rubber, chlorobutyl rubber, bromobutyl rubber, 1,3-butadiene/acrylonitrile copolymers, styrene/1,3-butadiene copolymers, styrene/isoprene/1,3-butadiene copolymers, and styrene/1,3-butadiene/acrylonitrile copolymers, and mixtures thereof.

29. The process according to claim 22, wherein the at least one reinforcing filler is selected from: sp² hybridized carbon allotropes, silica, layered silicates, mixed oxides of aluminium and magnesium with lamellar structure, alumina and silico aluminates, and mixtures thereof.

30. The process according to claim 29, wherein the sp² hybridized carbon allotropes are selected from: carbon black, graphene, 2-layer graphene, from 3 to 10 layer graphene, graphite, high surface area graphite, single wall or multiwall carbon nanotubes, carbon nanotubes of longitudinal or helicoid extension, nanocones, nanohorns, nanotoroids, fullerene, and mixtures thereof.

31. The process according to claim 30, wherein the sp² hybridized carbon allotropes are selected from: carbon black, graphene, graphite, high surface area graphite, single wall or multiwall carbon nanotubes, and mixtures thereof.

32. The process according to claim 22, wherein the at least one reinforcing filler is a sp² hybridized carbon allotrope present in an amount of at least 1 phr.

33. The process according to claim 30, wherein the sp² hybridized carbon allotrope is carbon black.

34. The process according to claim 33, wherein carbon black is present in an amount of at least 10 phr.

35. The process according to claim 22, wherein the at least one reinforcing filler is a mixture of carbon black and silica.

36. The process according to claim 35, wherein carbon black is present in an amount of at least 10 phr and silica is present in an amount equal to or lower than 100 phr.

37. The process according to claim 31, wherein the at least one reinforcing filler is present in an amount of from 10 phr to 150 phr.

38. The process according to claim 22, wherein one or more additives are further present in step a), and wherein the additives selected from anti-ageing agents, plasticizers, adhesives, modifying resins, coupling agents, and mixtures thereof.

39. The process according to claim 38, wherein the anti-ageing agents are selected from antioxidants, anti-ozone agents, and mixtures thereof.

40. The process according to claim 39, wherein in step a), at least one antioxidant or at least one anti-ozone agent is present.

41. The process according to claim 39, wherein the at least one antioxidant, the at least one anti-ozone agent, or both are each independently present in an amount of from 0.1 phr to 20 phr.

42. The process according to claim 22, wherein in step a), one or more vulcanizing agents, optionally comprising an activator, an accelerator, or both, are further present.
